(19) United States
(12) Patent Application Publication
(10) Pub. No.: US 2009/0240049 A1
(43) Pub. Date: Sep. 24, 2009

(54) Purification of air sensitive steroids

(76) Inventors: Marco Villa, Milano (IT); Donato Motolese, Milan (IT)

Correspondence Address: KENYON & KENYON LLP, ONE BROADWAY, NEW YORK, NY, 10004, US

(21) Appl. No.: 12/383,049

(22) Filed: Mar. 18, 2009

Related U.S. Application Data
(60) Provisional application No. 61/069,994, filed on Mar. 18, 2008.
(60) Provisional application No. 61/060,638, filed on Jun. 11, 2008.

(51) Int. Cl. C07J 75/00

(52) U.S. Cl. 540/66

(57) ABSTRACT

The present invention provides a purification method for air sensitive steroids, by reacting an amino acid or an analogue thereof with the 21-aldehyde oxidation product of such air sensitive steroids to form an adduct that is separated from the purified air sensitive steroids.

### CROSS REFERENCE TO RELATED APPLICATIONS

The present invention claims the benefit of the following U.S. Provisional Patent Applications Nos. 61/069,994, filed Mar. 18, 2008, and 61/060,638, filed Jun. 11, 2008. The contents of these applications are incorporated herein by reference.

### FIELD OF THE INVENTION

The present invention relates to a method for purifying air sensitive steroids from their corresponding oxidation impurities.

### BACKGROUND OF THE INVENTION

Steroids are a wide class of compounds including many different chemical entities. They represent one of the milestones in chemistry applied to health care of humans. Their structures share the skeleton of formula I.

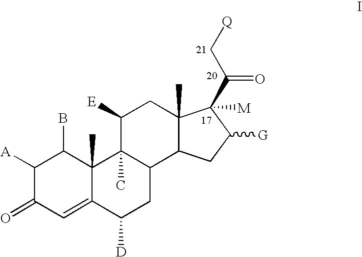

Table 1 lists several of the most commonly used Steroids.

TABLE 1

| Common name | A | B | C | D | E | G | M | Q |
|---|---|---|---|---|---|---|---|---|
| Betamethasone | Double | | F | H | OH | beta CH₃ | OH | OH |
| | bond | | | | | | | |
| Betamethasone propionate | Double | | F | H | OH | beta CH₃ | EtCOO | EtCOO |
| | bond | | | | | | | |
| Betamethasone acetate | Double | | F | H | OH | beta CH₃ | OH | MeCOO |
| | bond | | | | | | | |
| Betamethasone 17 valerate | Double | | F | H | OH | beta CH₃ | nBuCOO | OH |
| | bond | | | | | | | |
| Beclomethasone | Double | | Cl | H | OH | beta CH₃ | EtCOO | EtCOO |
| dipropionate | bond | | | | | | | |
| Diflorasone diacetate | Double | | F | F | OH | beta CH₃ | MeCOO | MeCOO |
| | bond | | | | | | | |
| Fludrocortisone acetate | H | H | F | H | OH | H | OH | MeCOO |

| Difluprednate | Double | F | F | OH | H | nPrCOO | MeCOO |
|---|---|---|---|---|---|---|---|
| | bond | | | | | | |
| Flumethasone pivalate | Double | F | F | OH | alpha CH₃ | OH | tBuCOO |
| | bond | | | | | | |
| Fluorometholone | Double | F | CH₃ | OH | H | OH | H |
| | bond | | | | | | |
| Fluorometholone 17 | Double | F | CH₃ | OH | H | MeCOO | H |
| acetate | bond | | | | | | |
| Deoxymethasone | Double | F | H | OH | alpha CH₃ | H | OH |
| | bond | | | | | | |
| Amcinonide | Double | F | H | OH | Ciclopentanone ketal | | MeCOO |
| | bond | | | | | | |
| Desonide | Double | H | H | OH | acetone ketal | | OH |
| | bond | | | | | | |
| Budesonide | Double | H | H | OH | nPr-CHO ketal | | OH |
| | bond | | | | | | |
| Flunisolide | Double | H | F | OH | Acetone ketal | | OH |
| | bond | | | | | | |
| Fluocinolone acetonide | Double | F | F | OH | Acetone ketal | | OH |
| | bond | | | | | | |
| Fluocinonide | Double | F | F | OH | Acetone ketal | | MeCOO |
| | bond | | | | | | |
| Triamcinolone acetonide | Double | F | H | OH | Acetone ketal | | OH |
| | bond | | | | | | |
| 16 alpha hydroxy | Double | H | H | OH | OH | OH | OH |
| prednisolone | bond | | | | | | |

Archiv for Pharmaci og Chemi, Scientific Edition 8, 1980, 187-206 and the Journal of Organic Chemistry, 1963, 28, 2001, describe the oxidation of the alcohol at the 21 carbon (“21-OH”) to the corresponding aldehyde (“21-CHO”). As reported by these references, the α-ketolic side chain of steroids is easily oxidized by air to give the corresponding 21-dehydro derivative (steroid-glyoxal), which undergoes further oxidative degradation. This oxidation takes place mostly under neutral or alkaline conditions and is catalyzed by traces of metals, such as iron.

Thus, it is difficult to control and/or avoid the formation of the 21-aldehyde impurity, which will eventually contaminate the steroid.

Impurities in steroids are undesirable and, in extreme cases, might even be toxic to a patient being treated with a dosage form containing the steroid. Because of the toxicity of the 21-aldehyde impurities of steroids, their established limit in the API is often stated to be less than 0.10%, see, for example, the 2008 U.S. Pharmacopoeia wherein the limit for 21-dehydro budesonide in Budesonide is defined as 0.07% (ref. USP 31, Vol. 2, p. 1565-6).

As known by those skilled in the art, the management of process impurities is greatly enhanced by understanding their chemical structures and synthetic pathways and by identifying the parameters that influence the amount of impurities in the final product.

Thus, methods that would allow for easy purification of steroids from their 21-aldehyde impurities as well as stabilize the steroids against further oxidation are needed. The present invention addresses this need, providing steroids free of 21-aldehyde oxidation products, and means for preparation thereof.

### SUMMARY OF THE INVENTION

In one embodiment, the present invention provides a method for purifying air-sensitive steroids of the following formula I

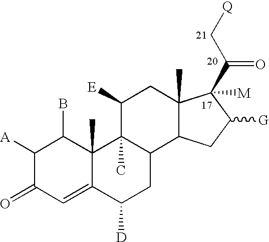

from the 21-aldehyde steroid impurity, in either hydrated or non-hydrated form, of the following formula,

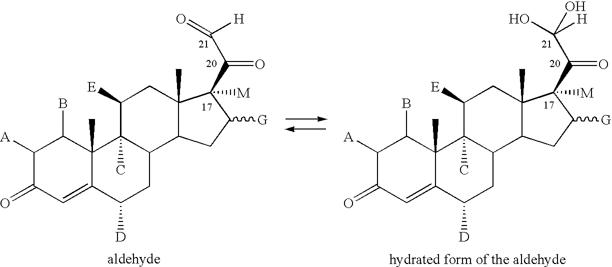

comprising combining an air-sensitive steroid with an amino acid or an analogue thereof of formula II:

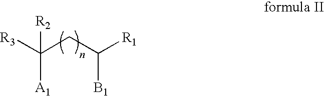

to obtain a mixture; and

recovering the air sensitive steroid from the mixture to obtain a purified air sensitive steroid,

wherein A and B are each H or together represent a double bond;
C is H, F, Cl, or OH;
D is H, CH₃, Cl, or F;
E is H, OH or a carbonyl;
G is H, OH, CH₃ or an oxygen atom that together with M forms a ketal or acetal with a C₁-C₆ linear or branched or cyclic carbonyl;
M is H, OH, an OH esterified with a C₁-C₆ linear or branched mono or bicarboxylic acid or with benzoic acid, or an oxygen atom that together with G forms a ketal or acetal with a C₁-C₆ linear or branched or cyclic carbonyl;
Q is OH or an OH esterified with a C₁-C₆ linear or branched carboxylic acid;
A₁ is SH or OH;
B₁ is NHR₂ or NHCOR_{4;}
R₁ is H, COOH or COR_{5;}
R₂, R₃ are each independently H, CH₃ or C₂H₅;
R₄ is a linear or branched C₁-C₄ alkyl;
R₅ is a peptide of the following formula:

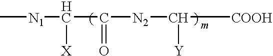

wherein X and Y are each independently substituents of an amino-acid; N₁ and
N₂ are each independently NH or N₁ forms together with X and/or N₂ forms together with Y an amino acid substituent; and
m=0 or 1 or 2; and
n=0 or 1.

In another embodiment, the present invention provides an adduct of formula III,

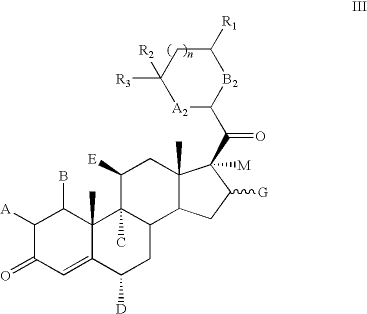

wherein
A₂=S or O;
B₂═NR₂ or NCOR₄;
A and B are each H or together represent a double bond;
C is H, F, Cl, or OH;
D is H, CH₃, Cl, or F;
E is H, OH or a carbonyl;
G is H, OH, CH₃ or an oxygen atom that together with M forms a ketal or acetal with a C₁-C₆ linear or branched or cyclic carbonyl;
M is H, OH, an OH esterified with a C₁-C₆ linear or branched mono or bicarboxylic acid or with benzoic acid, or an oxygen atom that together with G forms a ketal or acetal with a C₁-C₆ linear or branched or cyclic carbonyl;
R₁ is H, COOH or COR_{5;}
R₂, R₃ are each independently H, CH₃ or C₂H₅;
R₄ is a linear or branched C₁-C₄ alkyl;
R₅ is a peptide of the following formula:

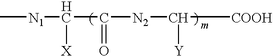

wherein X and Y are each independently substituents of an amino-acid; N₁ and
N₂ are each independently NH or N₁ forms together with X and/or N₂ forms together with Y an amino acid substituent; and
m=0 or 1 or 2; and
n=0 or 1.

### DETAILED DESCRIPTION OF THE INVENTION

The present invention provides a method for purifying steroids, especially air-sensitive steroids, from their corresponding oxidation impurity, a 21-aldehyde derivative.

During synthesis of these steroidal products, especially industrial synthesis, the following two scenarios occur:

1. Oxygen is present, even if an inert atmosphere is used during process operations for manufacturing the steroidal product, because of its solubility in water and in the organic solvents commonly used in the manufacturing processes. Common industrial process operations, wherein the steroid is exposed to air, are, for instance: filtration of the solid after crystallization, discharge from a Buchner funnel or from a centrifuge, loading into the vacuum oven, collection of the dried product and packaging, and storage in a closed container; in addition, if the product is micronized, the package is reopened and subsequent loading, discharging, and packaging may again occur in the presence of air. Moreover, micronization enlarges the surface area of the solid, thereby increasing possible exposure of the steroid in particles to air and to oxidation.

2. Traces of metals such as Chromium, Nickel, Molybdenum and Iron may be present in the raw materials or in the equipment that is used to prepare the steroidal product. These metals catalyze the oxidation of the steroid (see example 16).

Thus, the 21-aldehyde impurity that is difficult to purify from by conventional methods (for example, see examples 2 and 9), can be formed at any stage of the preparation, packaging and storage of the steroid.

Due to structure similarity between the aldehyde impurity and the steroid, there is a need for repetitive purification steps. However, this decreases the yield and can lead to the exposure of the product to oxygen, thereby possibly partially re-forming the 21-aldhyde impurity.

As is known in the field of steroids and their production, the term “air sensitive” in reference to steroids refers to steroids with an α-ketolic side chain (Q=OH in Table 1) or esters prepared from intermediates with an α-ketolic side chain, thereof of the following formula

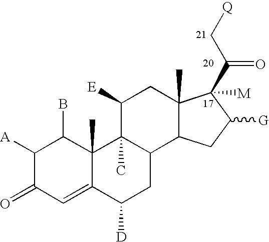

wherein A and B are each H or together represent a double bond;
C is H, F, Cl or OH;
D is H, CH₃, Cl or F;
E is H, OH or a carbonyl;
G is H, OH, CH₃ or an oxygen atom that together with M forms a ketal or acetal with a C₁-C₆ linear or branched or cyclic carbonyl;
M is H, OH, an OH esterified with a C₁-C₆ linear or branched mono or bicarboxylic acid or with benzoic acid or an oxygen atom that together with G forms a ketal or acetal with a C₁-C₆ linear or branched or cyclic carbonyl; and
Q is OH or an OH esterified with a C₁-C₆ linear or branched carboxylic acid,
   that, when exposed to even trace amounts of air, the 21-OH group (Q=OH) is oxidized, providing the 21-aldehyde steroid analogue. Examples of such steroids are listed in Table 1.

As known in the art, the term an “amino acid substituent” is the side-chain of an amino acid.

As used herein, the term “room temperature” refers to a temperature of about 20° C. to about 30° C., more preferably about 20° C. to about 25° C.

As used herein, the term “percent” or “%” refers to the percent area as measured by HPLC, unless otherwise indicated.

As used herein, dipolar aprotic solvent refers to a solvent lacking acidic hydrogens and containing at least one polarized bonds between carbon and a heteroatom, typically a multiple bond between carbon and either oxygen or nitrogen.

As used herein, apolar solvent refers to solvents having a low dielectric constant and not miscible with water.

In one embodiment, the present invention provides a method of purifying air-sensitive steroids of the following formula I

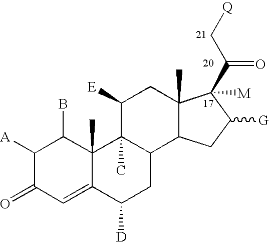

from the 21-aldehyde steroid impurity, in either hydrated or non-hydrated form, of the following formula,

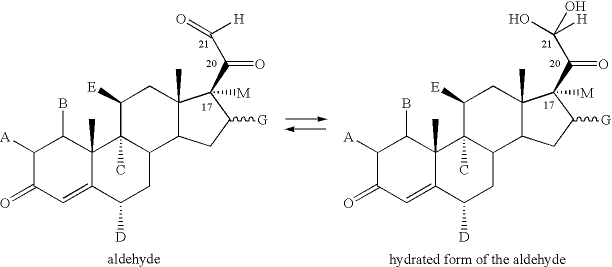

comprising combining an air-sensitive steroid with an amino acid or an analogue thereof of formula II:

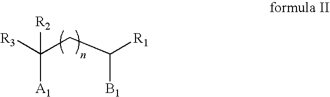

to obtain a mixture; and

recovering the air sensitive steroid from the mixture to obtain a purified air sensitive steroid,

wherein A and B are H or together represent a double bond;
C is H, F, Cl, or OH;
D is H, CH₃, Cl, or F;
E is H, OH a carbonyl;
G is H, OH, CH₃ or an oxygen atom that together with M forms a ketal or acetal with a C₁-C₆ linear or branched or cyclic carbonyl;
M is H, OH, an OH esterified with a C₁-C₆ linear or branched mono or bicarboxylic acid or with benzoic acid or an oxygen atom that together with G forms ketal or acetal with C₁-C₆ linear or branched or cyclic carbonyl compounds;
Q is OH or an OH esterified with a C₁-C₆ linear or branched carboxylic acid;
A₁ is SH or OH;
B₁ is NHR₂ or NHCOR_{4;}
R₁ is H, COOH, COR_{5;}
R₂, R₃ are each independently H, CH₃, C₂H₅;
R₄ is a C₁-C₄ linear or branched alkyl;
R₅ is a peptide of the following formula:

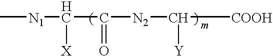

wherein X and Y are each independently substituents of an amino acid; N₁ and
N₂ are each independently NH or N₁ forms together with either X and/or N₂ forms together with Y an amino acid substituent; and
m=0,1 or 2; and
n=0 or 1.

Typically, G and M, N₁ and X, N₂ and Y are connected to each other directly or via at least one atom, thus forming together a ketal or acetal (G and M) or an amino acid substituent (N₁ and X, N₂ and Y), respectively.

Typically, the air-sensitive steroid is a steroid, such as any one of those listed in Table 1, which is contaminated with its corresponding 21-aldehyde impurity.

Preferably, the air-sensitive steroid is Flunisolide, 16-alpha-hydroxyprednisolone, Budesonide or Deoxymethasone. More preferably the steroid is 16-alpha-hydroxyprednisolone or Budesonide, most preferably the steroid is Budesonide.

Preferably, A₁ in formula II is SH.

Preferably, B₁ is NHR₂, more preferably NH₂.

Preferably, R₁ is COOH or COR₅, more preferably, COOH.

Preferably, R₂ and R₃ are H or CH₃, more preferably H.

Preferably, R₄ is CH₃

Preferably, X and Y are substituents of the following amino acids: Alanine, Asparagine, Aspartate, Arginine, Cysteine, Glutamate, Glutamine, Glycine, Histidine, Isoleicine, Leucine, Lysine, Methionine, Phenylalanine, Serine, Threonine, Tryptophane, Tyrosine and Valine and Proline.

Preferably, the substituent is a substituent of Glycine or Alanine, more preferably of Glycine.

The aldehyde can exist in the carbonylic form or the hydrated form, depending on the conditions, for example, the carbonylic form is more predominant under anhydrous conditions.

The amino acid or analogue thereof of formula II can be either optically or not optically active, i.e. not optically active is without a chiral center or a racemic mixure. Also commercial salts, such as hydrochloride salts, can be used as a source for the amino acid and analogues thereof, by reacting them with a base.

The amino acid or analogue thereof of formula II reacts with the aldehyde impurity (hydrated or in the carbonylic form) generating an adduct of formula III,

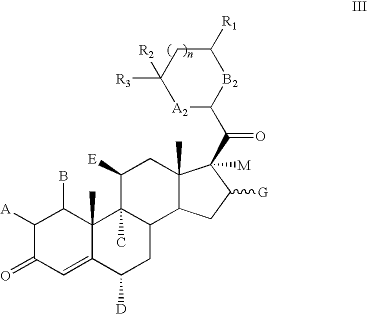

wherein

A₂=S or O;

B₂═NR₂ or NCOR₄; and

R₁, R₂, R₃, R₄, A, B, C, D, E, M and G are as described before, which can be easily removed by extractions and/or by crystallization, with minor loss of yield.

Further, the amino acid or analogue thereof of formula II forms complexes with the metals thus assisting in avoiding the catalysis of the oxidation reaction that is performed by the free form of the metal.

Preferably, A₂ in formula III is S.

The adduct of formula III wherein the steroid is Budesonide and the amino acid or analogue thereof of formula II is L-cysteine, prepared as a pure sample by conventional methods from the corresponding 21-aldehyde, is characterized by data selected from the group consisting of ¹H NMR (DMSO, 400.13 MHz, DMSO, 303° K): diastereoisomer 1: δ 7.301(d,1H), 6.158(dd, J 10.1,1.9,1H), 5.916(dd, J 1.9,1.2 1H), 2.518(m,1H), 2.287(dd,J 13.5,3.2 1H), 1.968(m,1H), 1.111(m,1H), 2.012(m, 1H), 1.027(dd, J 11.3, 3.4, 1H), 4.285(m, 1H), 1.864(dd, J 13.7, 3.7, 1H), 1.768(dd, J 13.7. 2.5, 1H), 1.569(m, 1H), 1.779(m, 1H), 1.601(m, 1H), 5.023(d, J 7.5, 1H), 0.865(s,3H), 1.375(s,3H), 4.872(d, J 4.0, 1H), 5.149(t, J 5.3, 1H), 1.595(m, 2H), 1.356(m, 2H), 0.875(t, J 7.4, 3H), 5.241(s, 1H), 2.974(d, J 5.6, 2H), 4.409 (t, J 5.6, 1H), 12.746(br s, 1H); diastereoisomer 2: 7.313(d, J 10.1 1H), 6.163(dd, J 10.1, 1.9, 1H), 5.917(dd, J 1.9,1.1,1H), 2.526(m, 1H), 2.294(ddd, J 13.5, 4.7, 1.7,1H), 2.001(m, 1H), 1.023(m, 1H), 2.089(m, 1H), 0.963(m, 1H), 4.134(m, 1H), 1.851(dd, J 13.5, 3.4 1H), 1.760(dd, J 13.5. 2.4, 1H), 1.554(m, 1H), 1.630(m, 1H), 1.519(m, 1H), 4.737(d, J 4.5, 1H), 0.815(s, 3H), 1.381(s, 3H), 4.929(d, J 4.1, 1H), 4.809(t, J 4.6, 1H), 1.546(m, 2H), 1.364(m, 2H), 0.874(t, J 7.4, 3H), 5.266(s, 1H), 3.020(dd, J 10.2, 4.2, 1H), 2.957(dd, J 10.2, 6.6, 1H), 4.424(dd, J 6.6, 4.2, 1H), 12.835(br, s, 1H); and LC/MS: at m/z 532 the molecular ion is present.

Preferably, the amino acid or analogue thereof of Formula II is chosen from the list consisting of L-cysteine, cysteamine, penicillamine, cysteyl-glycine (Cys-Gly), homocysteine and L-serine, as depicted in the following table.

| Compound | Structure |
|---|---|
| L-cysteine | 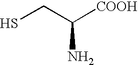 |
| cysteamine | 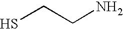 |
| DL-penicillamine | 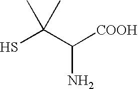 |
| Cys-Gly | 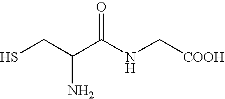 |
| DL-homocysteine | 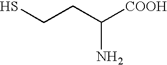 |
| L-serine | 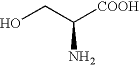 |

The preferred amino acid or an analogue thereof of Formula II is L-cysteine.

Preferably, the purification comprises combining the air-sensitive steroid, an amino acid or an analogue thereof of formula II and a solvent selected from the group consisting of an alcohol, an ester, a ketone, a haloalkane, a dipolar aprotic solvent, a nitrile, an ether, and mixtures thereof, and mixtures thereof with water, to obtain a solution also comprising the adduct of formula III, from which the air sensitive steroid is recovered to obtain such purified steroid.

The amino acid or analogue thereof of formula II is present in the solution in sufficient excess to remove the 21-aldehyde impurity. Preferably, the mole ratio of the amino acid or analogue thereof of formula II to aldehyde is from about 2:1 to about 10:1, more preferably it is from about 3:1 to about 5:1, most preferably it is about 4:1, respectively.

Preferably, the alcohol is a linear or branched C₁-C₅ alcohol, more preferably a C₁-C₄ alcohol, even more preferably methanol, ethanol, isopropanol or isobutanol, most preferably methanol.

Preferably, the ester is a C₂-C₅ ester, more preferably a C₃-C₅ ester, most preferably ethyl acetate.

Preferably, the ketone is C₂-C₅ ketone, more preferably acetone, butanone or isopropyl methyl ketone, most preferably acetone.

Preferably, the haloalkane is a C₁-C₅ haloalkane, more preferably a C₁-C₂ haloalkane, most preferably dichloromethane.

Preferably, the dipolar aprotic solvent is a C₂-C₅ dipolar aprotic solvent, more preferably dimethylsulfoxide (DMSO), dimethylformamide (DMF) or dimethylacetamide (DMA), most preferably dimethylformamide.

Preferably the nitrile is a C₁-C₅ nitrile, more preferably C₁-C₃ nitrile, most preferably acetonitrile.

Preferably, the ether is a C₄-C₆ ether, more preferably tetrahydrofuran, 2-methyltetrahydrofuran or dioxane, most preferably tetrahydrofuran.

Preferably, mixtures with water comprise an alcohol, a haloalkane and water, a dipolar aprotic solvent and water, an ether and water, a ketone and water, an alcohol and water, more preferably, the mixture is a mixture of methanol, dichloromethane and water, DMF and water, THF and water, dioxane and water, methanol and water or acetone and water, most preferably, the mixture is a mixture of methanol, dichloromethane and water.

Optionally, the solution can contain an acid, which can catalyze the formation of the adduct in the reaction of the aldehyde with the amino acid or analogue thereof of formula II. Preferably, the acid is an organic acid, more preferably, acetic acid, formic acid or paratoluene sulfonic acid (PTSA), most preferably, acetic acid.

Preferably, the mole ratio of catalyst:amino acid or analogue thereof of formula II is about 1:10 to about 5:1, more preferably 4:1, even more preferably 2:1.

Combining the air sensitive steroid and the solvent can provide a solution or a suspension, depending on the solvent.

To aid in dissolution, the suspension can be heated. Preferably, the dissolution is performed at a temperature of about room temperature to about 70° C., more preferably at about 40° C. to about 60° C., most preferably at about 60° C. depending on the solvent. Preferably, the dissolution is performed for about 30 minutes to about 2 hours, more preferably for about one hour.

The completion of the reaction can be determined by HPLC, by monitoring the increase in the amount of the adduct or the disappearance of the aldehyde.

Preferably, the concentration of the air sensitive steroid in the solvent is from about 10 g/l to about 200 g/l, more preferably it is about 100 g/l.

Prior to recovering the steroid, the reaction mixture can be concentrated. Preferably, the reaction mixture is a solution.

The recovery of the purified steroid may be done by separating the air sensitive steroid from the adduct of formula III. Preferably, the recovery can be done either by precipitation of the purified steroid from the reaction solution, followed by separation of it from the remaining soluble adduct to obtain a purified steroid, or by extracting the obtained adduct from the reaction solution.

Preferably, the precipitation is done by combining the reaction solution with an anti-solvent to obtain a second mixture, optionally followed by either cooling or by concentrating and cooling the obtained second mixture.

Preferably, the anti-solvent is water.

Preferably, concentration is done at a temperature of about 40° C. to about 80° C., more preferably of about 60° C. to about 70° C., most preferably at about 70° C.

Preferably, cooling is done to a temperature of about room temperature to about 0° C., more preferably, to about 0° C.

The precipitated purified steroid can then be filtered, i.e., separated from remaining solution containing the adduct of formula III, followed by washing the filtered purified steroid.

Preferably, the extraction is performed by combining the reaction solution with an acidic or basic aqueous solution; separating the obtained phases, wherein the organic phase contains the purified steroid and the aqueous phase contains the adduct and/or its decomposition products and the base or acid; washing the organic phase with water, and isolating the purified steroid from the organic phase. Preferably, the isolation can be done by either removing the solvent or by crystallization.

Preferably, the basic aqueous solution is an aqueous solution of sodium carbonate or sodium hydrogen carbonate, more preferably of sodium hydrogen carbonate.

Preferably, the pH of the basic aqueous solution is greater than 7, more preferably it is greater than 7 to about 8.

Preferably, the pH of the acidic aqueous solution is less than 7, more preferably it is less than 7 to about 5, most preferably less than 7 to about 6. Preferably, the acid is HCl.

Preferably, the crystallization is done by combining the organic phase with an anti-solvent, thus providing a suspension comprising the crystalline steroid. The precipitated steroid can then be filtered from the suspension.

Preferably, the anti-solvent is water or a C₄-C₈ apolar solvent, more preferably the apolar solvent is a C₅-C₈ alkane or a C₄-C₆ ether. Preferably the C₅-C₈ alkane is hexane or heptane. Preferably, the C₄-C₆ ether is diisopropyl ether or diethyl ether or methyl tertbutyl ether. Most preferably the anti-solvent is water.

Preferably, the levels of the 21-aldehyde impurity in the purified steroid obtained by the purification process of the invention is less than or equal to about 0.15%, more preferably less than or equal to about 0.10%, most preferably less than or equal to about 0.01%, for example, between about 0.005% and about 0.10%, between about 0.008% and about 0.07%, between about 0.009% and about 0.05%.

Having described the invention with reference to certain preferred embodiments, other embodiments will become apparent to one skilled in the art from consideration of the specification. The invention is further defined by reference to the following examples describing in detail the purification of the air sensitive steroid using an amino acid or analogue thereof. It will be apparent to those skilled in the art that many modifications, both to materials and methods, may be practiced without departing from the scope of the invention

### EXAMPLES

### Analytical Methods Used

### HPLC

The amount of air-sensitive steroid and/or its 21 aldehyde impurity were determined using HPLC. Common HPLC methods for these steroids and their 21-aldehyde impurities may be found in various monographs see for example for Budesonide and its 21-aldehyde impurity a HPLC method is reported in USP31-NF26, page 1565 (detection limit 0.02%), for Fluocinolone acetonide a HPLC method in European Pharmacopoeia (EP) 6.0, page 1915, and for Methylprednisolone a HPLC method in European Pharmacopoeia (EP) 6.0, page 2393.

### Mass Spectrometry

Instrument: Finnigan LCQ (ion-trap)
Method: ESI, positive ion mode
Conditions: Source Voltage (kV) 3.50, Source Current (μA) 1.85, Capillary Voltage (V) 17.86, Capillary Temp (° C.): 190.00. Mobile phase 10 mM ammonium fornate in 70% aqueous methanol.

### Example 1

### Purification of Flunisolide With cysteine

20 g Flunisolide (6α-fluoro-11β,6α, 17, 21-tetrahydroxy-pregna-1,4-dien-3,20-dione 16,17-acetonide) (0.046 mol) containing 0.15% 21-dehydro impurity (aldehyde) were dissolved in 225 ml acetone at 40° C. 27 mg L-cysteine (0.22 mmol) were added and the solution was left stirring for 1 hour. The mixture was then poured into water (500 ml), concentrated under vacuum, cooled to room temperature, filtered and washed with water. The crystallized product contained 0.09% dehydro derivative (the 21-aldehyde).

### Comparative Example 2

### Purification of Flunisolide Without cysteine

20 g Flunisolide (6α-fluoro-11β,16α, 17, 21-tetrahydroxy-pregna-1,4-dien-3,20-dione 16,17-acetonide) (0.046 mol) containing 0.15% 21-dehydro impurity (aldehyde) were dissolved in 225 ml acetone at 40° C. and the solution was left stirring for 1 hour. The mixture was then poured into water (500 ml), concentrated under vacuum, cooled to room temperature, filtered and washed with water. The crystallized product contained 0.15% dehydro derivative (the 21-aldehyde): no purification occurred.

### Example 3

### Purification of Flunisolide With cysteine

25 g Flunisolide (6α-fluoro-11β,16α, 17, 21-tetrahydroxy-pregna-1,4-dien-3,20-dione 16,17-acetonide) (0.057 mol) containing 0.15% 21-dehydro impurity (aldehyde) were dissolved in 350 ml methanol at 40° C. 40 mg L-cysteine (0.33 mmol) were added and the solution was allowed to stir at 40° C. for 1 hour. Water was added (585 ml) and crystallization occurred; the mixture was cooled to 0° C., the solid was filtered and washed. The crystallized product contained 0.07% dehydro derivative (the 21-aldehyde).

### Example 4

### Purification of 16-alpha-hydroxyprednisolone With L-cysteine

20 g of 16-alpha-hydroxyprednisolone (0.053 mol) were dissolved at 40° C. in a mixture of 80 ml methanol, 40 ml dichlorometane and 40 ml water. This solution was found to contain 0.11% (HPLC area) of 21-dehydro compound (aldehyde). Then 30 mg L-cysteine (0.25 mmol) were added and the solution was allowed to stir at 40° C. for 1 hr. The treated solution was found to contain a reduced amount of 21-dehydroderivative (0.03% HPLC area). After concentration under vacuum and water addition, the product was filtered and washed. The crystallized product contained <0.01% 21-dehydro derivative (the 21-aldehyde).

### Example 5

### Purification of Budesonide With L-cysteine

20 g Budesonide (0.046 mol) containing 0.20% 21-dehydro derivative (HPLC area) and 50 mg L-cysteine (0.41 mmol) were dissolved in 250 ml methanol at 60° C. After stirring 1 hour at room temperature the product was crystallized by addition of 420 ml water. The mixture was then cooled to 0° C., the solid was filtered, washed with water and analyzed by HPLC: the 21-dehydro derivative (the 21-aldehyde) was not detected.

### Example 6

### Purification of Deoxymethasone With N-acetyl-cysteine

15 g of Deoxymethasone (9-Fluoro-11-beta, 21-dihydroxy-16-alpha-methylpregna-1,4-diene-3,20-dione) (0.040 mol) containing 0.61% 21-dehydro derivative (HPLC area) were dissolved in 150 ml dichloromethane and treated with 75 mg N-acetyl-cysteine (0.46 mmol). After stirring at room temperature for 30 minutes, the solution was washed with sodium hydrogen carbonate 2% in water (100 ml), then with water (50 ml). The organic phase was concentrated under vacuum, added of 75 ml methanol, concentrated at 70° C. to a reduced volume, cooled and finally treated with 150 ml water to crystallize the product. The solid was filtered, dried and analyzed by HPLC: the content of 21-dehydro derivative (the 21-aldehyde) was 0.28%.

### Example 7

### Purification of Deoxymethasone With D,L-cysteine

15 g of Deoxymethasone (9-Fluoro-11-beta, 21-dihydroxy-16-alpha-methylpregna-1,4-diene-3,20-dione) (0.040 mol) containing 0.61% 21-dehydro derivative (HPLC area) were dissolved in 150 ml dichloromethane and treated with 102 mg D,L-cysteine (0.84 mmol). After stirring at room temperature for 30 minutes, the solution was washed water (100 ml), then with aqueous sodium hydrogen carbonate (100 ml), then with water (150 ml). The organic phase was concentrated under vacuum, added of 75 ml methanol, concentrated at 70° C. to a reduced volume, cooled and finally treated with 150 ml water to crystallize the product. The solid was filtered, dried and analyzed by HPLC: the content of 21-dehydro derivative (the 21-aldehyde) was 0.06%.

### Example 8

### Purification of Deoxymethasone With D,L-cysteine

15 g of Deoxymethasone (9-Fluoro-11-beta, 21-dihydroxy-16-alpha-methylpregna-1,4-diene-3,20-dione) (0.040 mol) containing 0.19% 21-dehydro derivative (HPLC area) were dissolved in 60 ml methanol and treated with 100 mg D,L-cysteine (0.83 mmol). After stirring at 45° C. for 30 minutes, the solvent was distilled off to small volume. The residue was cooled to room temperature, dissolved in dichloromethane (150 ml), washed with aqueous sodium hydrogen carbonate (150 ml), then with water (150 ml). The organic phase was concentrated under vacuum, was added of 75 ml methanol, concentrated at 70° C. to a reduced volume, cooled and finally treated with 150 ml water to crystallize the product. The solid was filtered, dried and analyzed by HPLC: the content of 21-dehydro derivative (the 21-aldehyde) was 0.02%.

### General Procedure: Examples 9-15

### Purification of Budesonide With and Without cysteine-analogues

1 g Budesonide (“Budesonide start”) was mixed with 0.05 g of the corresponding 21-aldehyde in 12.5 ml methanol resulting in Budesonide having 0.36% of the aldehyde impurity; the cysteine-analogue was added in a quantity of 4 moles/mole aldehyde. After 1 h at 60° C., 21 ml water were added, the mixture was cooled to 0° C., the product filtered, washed with water, dried and analyzed by HPLC.

### Comparative Example 9

### Purification of Budesonide Without L-cysteine

1 g Budesonide (2.32 mmol) containing 0.36% 21-dehydro derivative (HPLC area) was suspended in 12.5 ml methanol at 60° C. After heating the suspension turned into a solution. After 1 hour the product was crystallized by addition of 21 ml water. The mixture was then cooled to 0° C., the solid was filtered, washed with water and analyzed by HPLC: the 21-dehydro derivative was 0.29%.

### Example 10

### Purification of Budesonide With L-cysteine

1 g Budesonide (2.32 mmol) containing 0.36% 21-dehydro derivative (HPLC area) and 6 mg L-cysteine (0.049 mmol) were suspended in 12.5 ml methanol at 60° C. After heating the suspension turned into a solution. After 1 hour the product was crystallized by addition of 21 ml water. The mixture was then cooled to 0° C., the solid was filtered, washed with water and analyzed by HPLC: the 21-dehydro derivative was 0.07%.

### Example 11

### Purification of Budesonide With DL-penicillamine

1 g Budesonide (2.32 mmol) containing 0.36% 21-dehydro derivative (HPLC area) and 8 mg DL-penicillamine (0.054 mmol) were suspended in 12.5 ml methanol at 60° C. After heating the suspension turned into a solution. After 1 hour the product was crystallized by addition of 21 ml water. The mixture was then cooled to 0° C., the solid was filtered, washed with water and analyzed by HPLC: the 21-dehydro derivative was 0.04%.

### Example 12

### Purification of Budesonide With cys-gly

1 g Budesonide (2.32 mmol) containing 0.36% 21-dehydro derivative (HPLC area) and 12 mg cys-gly (0.057 mmol) were suspended in 12.5 ml methanol at 60° C. After heating the suspension turned into a solution. After 1 hour the product was crystallized by addition of 21 ml water. The mixture was then cooled to 0° C., the solid was filtered, washed with water and analyzed by HPLC: the 21-dehydro derivative was not detected.

### Example 13

### Purification of Budesonide With DL-homocysteine

1 g Budesonide (2.32 mmol) containing 0.36% 21-dehydro derivative (HPLC area) and 7.7 mg DL-homocysteine (0.054 mmol) were suspended in 12.5 ml methanol at 60° C. After heating the suspension turned into a solution. After 1 hour the product was crystallized by addition of 21 ml water. The mixture was then cooled to 0° C., the solid was filtered, washed with water and analyzed by HPLC: the 21-dehydro derivative was 0.03%.

### Example 14

### Purification of Budesonide With L-serine

1 g Budesonide (2.32 mmol) containing 0.36% 21-dehydro derivative (HPLC area) and 7 mg L-serine (0.067 mmol) were suspended in 12.5 ml methanol at 60° C. After heating the suspension turned into a solution. After 1 hour the product was crystallized by addition of 21 ml water. The mixture was then cooled to 0° C., the solid was filtered, washed with water and analyzed by HPLC: the 21-dehydro derivative was 0.08%.

### Example 15

### Purification of Budesonide With cysteamine and Acetic Acid

1 g Budesonide (2.32 mmol) containing 0.36% 21-dehydro derivative (HPLC area), 4.3 mg cysteamine (0.053 mmol) and 1.1 mg acetic acid (0.017 mmol) were suspended in 12.5 ml methanol at 60° C. After heating the suspension turned into a solution. After 1 hour the product was crystallized by addition of 21 ml water. The mixture was then cooled to 0° C., the solid was filtered, washed with water and analyzed by HPLC: the 21-dehydro derivative was 0.12%.

### Example 16

### Stabiliy of Budesonide

Budenoside from three production lots prepared in stainless steel reactors was compared to a lot of Budenoside produced in such a reactor and purified with cysteine according to example 5. All samples were stored in double plastic containers. The samples that were not treated with cysteine were kept at a temperature of 25±2° C. and relative humidity of 60%. The sample treated with cysteine was stored at a temperature that varied from about 0° C. to about 40° C. with no humidity control. As is shown in the table below the amount of the 21-aldehyde impurity in the Budenoside purified according to example 5 is less than 0.07% even after 6 years of storage. The aldehyde content of all samples was measured by HPLC.

| | | | | | | | Budenoside purified | |
|---|---|---|---|---|---|---|---|---|
| | Budenoside | | Budenoside | | Budenoside | | according to example 5 | |
| Time | HPLC Area % | | HPLC Area % | | HPLC Area % | | HPLC Area % | |
| (Years) | 21 Aldehyde | Budenoside | 21 Aldehyde | Budenoside | 21 Aldehyde | Budenoside | 21 Aldehyde | Budenoside |
| 0 | nd | 99.0 | nd | 99.4 | nd | 99.2 | nd | 99.76 |
| 1 | nd | 98.8 | nd | 98.9 | nd | 99.0 | | |
| 2 | 0.1 | 98.7 | 0.6 | 98.5 | <0.1 | 99.1 | | |
| 5 | 0.2 | 98.4 | 0.9 | 97.8 | <0.2 | 98.2 | | |
| 6 | | | | | | | 0.02 | 99.61 |

nd = not detected

What is claimed:

1. A process for purifying air-sensitive steroids of formula I 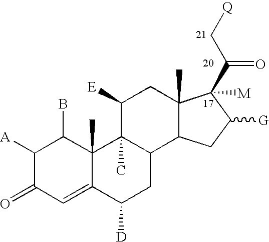
from an 21-aldehyde steroid impurity, in either hydrated or non-hydrated form, of the following formula, 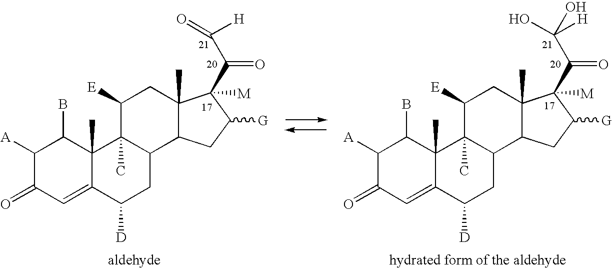
comprising
combining an air-sensitive steroid with an amino acid or analogue thereof of formula II: 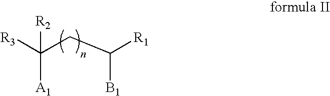
to obtain a mixture; and
recovering the air-sensitive steroid from the mixture to obtain a purified air sensitive steroid,
wherein A and B are each H or together represent a double bond;
C is H, F, Cl, or OH;
D is H, CH₃, Cl, or F;
E is H, OH or a carbonyl;
G is H, OH, CH₃ or an oxygen atom that together with M forms a ketal or acetal with a C₁-C₆ linear or branched or cyclic carbonyl;
M is H, OH, an OH esterified with a C₁-C₆ linear or branched mono or bicarboxylic acid or with benzoic acid, or an oxygen atom that together with G forms a ketal or acetal with a C₁-C₆ linear or branched or cyclic carbonyl;
Q is OH or an OH esterified with a C₁-C₆ linear or branched carboxylic acid;
A₁ is SH or OH;
B₁ is NHR₂ or NHCOR_{4;}
R₁ is H, COOH or COR_{5;}
R₂, R₃ are each independently H, CH₃ or C₂H₅;
R₄ is a linear or branched C₁-C₄ alkyl;
R₅ is a peptide of the following formula: 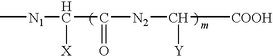
wherein X and Y are each independently substituents of an amino-acid; N₁ and
N₂ are each independently NH or N₁ forms together with X and/or N₂ forms together with Y an amino acid substituent; and
m=0 or 1 or 2; and
n=0 or 1.

2. The process of claim 1, wherein the air-sensitive steroid is chosen from the list consisting of Betamethasone, Betamethasone propionate, Betamethasone acetate, Betamethasone 17 valerate, Beclomethasone dipropionate, Diflorasone diacetate, Fludrocortisone acetate, Difluprednate, Flumethasone pivalate, Fluorometholone, Fluorometholone 17 acetate, Deoxymethasone, Amcinonide, Desonide, Budesonide, Flunisolide, Fluocinolone acetonide, Fluocinonide, Triamcinolone acetonide, and 16-alpha-hydroxyprednisolone.

3. The process of claim 2, wherein the air-sensitive steroid is chosen from the list consisting of Flunisolide, 16-alpha-hydroxyprednisolone, Budesonide and Deoxymethasone.

4. The process of claim 1 wherein the air-sensitive steroid is a steroid which is contaminated with its corresponding 21-aldehyde impurity.

5. The process of claim 1, wherein A in formula II is SH.

6. The process of claim 1 wherein B₁ in formula II is NHR₂.

7. The process of claim 6 wherein B₁ in formula II is NH₂.

8. The process of claim 1 wherein, R₁ in formula II is COOH or COR₅.

9. The process of claim 8 wherein R₁ in formula II is COOH.

10. The process of claim 1 wherein R₂ and R₃ in formula II are H or CH₃.

11. The process of claim 10 wherein R₂ and R₃ in formula II are H.

12. The process of claim 1 wherein, R₄ in formula II is CH₃.

13. The process of claim 1 wherein X and Y are each independently substituents of an amino-acid selected from the list consisting of: Alanine, Asparagine, Aspartate, Arginine, Cysteine, Glutamate, Glutamine, Glycine, Histidine, Isoleicine, Leucine, Lysine, Methionine, Phenylalanine, Serine, Threonine, Tryptophane, Tyrosine and Valine and Proline.

14. The process of claim 13, wherein X and Y are each independently substituents of Alanine or Glycine.

15. The process of claim 14, wherein X and Y are substituents of Glycine.

16. The process of claim 1 wherein the amino acid or an analogue thereof of formula II is selected from the list consisting of cysteine, cysteamine, penicillamine, cysteyl-glycine (Cys-Gly), homocysteine and L-serine.

17. The process of claim 16 wherein the amino acid or an analogue thereof is cysteine.

18. The process of claim 17 wherein the cysteine is L-cysteine.

19. The process of claim 1 wherein the purification comprises combining the air-sensitive steroid, an amino acid or an analogue thereof of formula II and a solvent to obtain a reaction solution from which the air-sensitive steroid is recovered to obtain a purified steroid.

20. The process of claim 19 wherein the solvent is selected from the group consisting of an alcohol, an ester, a ketone, a haloalkane, a dipolar aprotic solvent, a nitrile, an ether and mixtures thereof with water.

21. The process of claim 20 wherein the alcohol is a linear or branched C₁-C₅ alcohol, the ester is a C₂-C₅ ester, the ketone is a C₂-C₅ ketone, the haloalkane is a C₁-C₅ haloalkane, the dipolar aprotic solvent is a C₂-C₅ dipolar aprotic solvent, the nitrile is a C₁-C₅ nitrile and the ether is a C₄-C₆ ether.

22. The process of claim 21 wherein the C₁-C₅ alcohol is a linear or branched C₁-C₄ alcohol, the C₂-C₅ ester is a C₃-C₅ ester, the C₁-C₅ haloalkane is a C₁-C₂ haloalkane and the C₁-C₅ nitrile is a C₁-C₃ nitrile.

23. The process of claim 22 wherein the linear or branched C₁-C₄ alcohol is selected from the list consisting of methanol, ethanol, isopropanol and isobutanol, the C₂-C₅ ketone is selected from the list consisting of acetone, butanone, methyl ethyl ketone and isopropyl methyl ketone, the C₂-C₅ dipolar aprotic solvent is selected from the list consisting of dimethylsulfoxide (DMSO), dimethylformamide (DMF) or dimethylacetamide (DMA) and the C₄-C₆ ether is selected from the list consisting of tetrahydrofurane (THF), 2-methyltetrahydrofurane and dioxane.

24. The process of claim 23 wherein the C₁-C₄ alcohol is methanol, the C₃-C₅ ester is ethyl acetate, the C₂-C₅ ketone is acetone, the C₁-C₂ haloalkane is dichloromethane, the C₂-C₅ dipolar aprotic solvent is DMF, the C₂-C₅ nitrile is acetonitrile and the C₄-C₆ ether is THF.

25. The process of claim 20 wherein the mixture with water is selected from the list consisting an alcohol, a haloalkane and water, a dipolar aprotic solvent and water, an ether and water, a ketone and water and alcohol and water,

26. The process of claim 25 wherein the mixture is selected from the list consisting of mixtures of methanol, dichloromethane and water, DMF and water, THF and water, dioxane and water, methanol and water and acetone and water.

27. The process of claim 26 wherein the mixture is a mixture of methanol, dichloromethane and water.

28. The process of claim 20 wherein the solution further contains a catalyst.

29. The process of claim 28 wherein the catalyst is an organic acid selected from the list consisting of is acetic acid, formic acid or paratoluene solfonic acid (PTSA).

30. The process of claim 20 wherein the recovery of the air-sensitive steroid is done by separating the air-sensitive steroid from the adduct of formula III 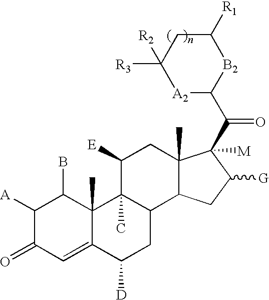
wherein
A₂=S or O;
B₂═NR₂ or NCOR₄;
A and B are each H or together represent a double bond;
C is H, F, Cl, or OH;
D is H, CH₃, Cl, or F;
E is H, OH or a carbonyl;
G is H, OH, CH₃ or an oxygen atom that together with M forms a ketal or acetal with a C₁-C₆ linear or branched or cyclic carbonyl;
M is H, OH, an OH esterified with a C₁-C₆ linear or branched mono or bicarboxylic acid or with benzoic acid, or an oxygen atom that together with G forms a ketal or acetal with a C₁-C₆ linear or branched or cyclic carbonyl;
R₁ is H, COOH, COR_{5;}
R₂, R₃ are each indepently H, CH₃, C₂H₅;
R₄ is a C₁-C₄ linear or branched alkyl;
R₅ is a peptide of the following formula: 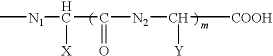
wherein X and Y are each independently substituents of an amino acid; N₁ and
N₂ are each independently NH or N₁ forms together with either X and/or N₂ forms together with Y an amino acid substituent; and
m=0,1 or 2;
n=0 or 1.

31. The process of claim 30 wherein prior to recovering the steroid, the reaction solution is concentrated.

32. The process of claim 30 wherein the recovery is done by precipitation of the purified steroid from the reaction solution, followed by separation of the purified steroid from the remaining soluble adduct, or by extracting the obtained adduct from the reaction solution.

33. The process of claim 32 wherein the precipitation is done by combining the reaction solution with an anti-solvent to obtain a second mixture.

34. The process of claim 32 wherein the extraction is performed by combining the reaction solution with an acidic or basic aqueous solution; separating the obtained phases; washing the organic phase with water, and isolating the purified steroid from the organic phase.

35. The process of claim 34 wherein the basic aqueous solution is an aqueous solution of sodium carbonate or sodium hydrogen carbonate.

36. The process of claim 35 wherein the basic aqueous solution is an aqueous solution of sodium hydrogen carbonate.

37. The process of claim 36 wherein the pH of the basic aqueous solution is greater than 7.

38. The process of claim 34 wherein the pH of the acidic aqueous solution is less than 7.

39. The process of claim 34 wherein isolation is done by precipitation after the addition of an anti-solvent to crystallize it.

40. The process of claim 39 wherein the anti-solvent is water, or a C₄-C₈ apolar solvent.

41. The process of claim 40 wherein the C₄-C₈ apolar solvent is a C₅-C₈ alkane or a C₄-C₆ ether.

42. The process of claim 41 wherein the C₅-C₈ alkane is hexane or heptane and the C₄-C₆ ether is diisopropyl, diethyl ether, or methyl tertbutyl ether.

43. The process of claim 42 wherein the anti-solvent is water.

44. The adduct of formula III 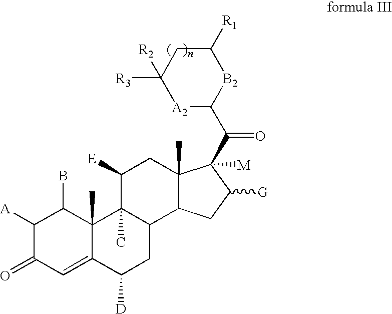
wherein
A₂=S or O;
B₂═NR₂ or NCOR₄;
A and B are each H or together represent a double bond;
C is H, F or OH;
D is H, CH₃ or F;
E is H, OH or a carbonyl;
G is H, OH, CH₃ or an oxygen atom that together with M forms a ketal or acetal with a C₁-C₆ linear or branched or cyclic carbonyl;
M is H, OH, an OH esterified with a C₁-C₆ linear or branched mono or bicarboxylic acid or with benzoic acid, or an oxygen atom that together with G forms a ketal or acetal with a C₁-C₆ linear or branched or cyclic carbonyl;
Q is OH or an OH esterified with a C₁-C₆ linear or branched carboxylic acid;
R₁ is H, COOH or COR_{5;}
R₂, R₃ are each independently H, CH₃ or C₂H₅;
R₄ is a linear or branched C₁-C₄ alkyl;
R₅ is a peptide of the following formula: 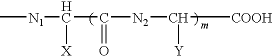
wherein X and Y are each independently substituents of an amino-acid; N₁ and
N₂ are each independently NH or N₁ forms together with X and/or N₂ forms together with Y an amino acid substituent; and
m=0 or 1 or 2; and
n=0 or 1.

45. The adduct of claim 44 wherein A₂ is S.
